(19) United States
(12) Patent Application Publication
(10) Pub. No.: US 2019/0201846 A1
(43) Pub. Date: Jul. 4, 2019

(54) APPARATUS, SYSTEMS, AND METHODS FOR ISOTOPE EXCHANGE AND/OR DIALYSIS

(71) Applicant: University of Kansas, Lawrence, KS (US)

(72) Inventors: David D. Weis, Lawrence, KS (US); Mihiri Shyamali Weerasinghe, Lawrence, KS (US)

(21) Appl. No.: 16/235,442

(22) Filed: Dec. 28, 2018

Related U.S. Application Data
(60) Provisional application No. 62/612,381, filed on Dec. 30, 2017.

(51) Int. Cl. B01D 61/28; G01N 1/40

(52) U.S. Cl. CPC B01D 61/28; A61M 1/16; G01N 2001/4016; G01N 1/4005

(57) ABSTRACT

A dialysis and/or isotope exchange device includes a support, a sample chamber having a thickness of between 25 μm and 500 μm, and a container including a semipermeable membrane in contact with the sample chamber. Dialysis and/or isotope exchange takes less than 100,000 seconds for a concentration of a substance to reach a target concentration.

### CROSS-REFERENCE TO RELATED APPLICATIONS

This application claims priority to and the benefit of U.S. Provisional Patent Application No. 62/612,381 filed on Dec. 30, 2017, which is hereby incorporated by reference in its entirety.

### BACKGROUND

### Background and Relevant Art

Dialysis involves the transfer of a substance across a semipermeable membrane to increase or decrease the concentration of one or more substances in a sample. Dialysis is often dependent upon diffusion to change the concentration of the substance. Dialysis may have many applications, including medical diagnosis, medical research, chemical research, physical science research, and other applications. Many of these applications are time-sensitive, and dialysis can be a bottle-neck in the application.

### BRIEF SUMMARY

In some embodiments, an isotope exchange device may include a support, a sample container on the support, a container with a semipermeable membrane on one end, and a sample chamber having a thickness of between 100 μm and 500 μm.

In other embodiments, a dialysis device may include a sample placed in a sample chamber, a dialysate placed in a container, and a semipermeable membrane on one end of the dialysate container. The sample and the dialysate may have different concentrations of one or more substances.

In still other embodiments, a method for dialysis may include placing a sample having a first concentration of a substance in a sample chamber, placing a container on top of the sample chamber, the container including a dialysate having a second concentration of the substance, and waiting for the first concentration to reach a target concentration.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1-1 is a side cutaway view of a dialysis device, according to at least one embodiment of the present disclosure;

FIG. 1-2 is a close-up cutaway view of a portion of the dialysis device of FIG. 1-1, according to at least one embodiment of the present disclosure;

FIG. 2-1 is a side cutaway view of another embodiment of a dialysis device, according to at least one embodiment of the present disclosure;

FIG. 2-2 is a side cutaway view of still another embodiment of a dialysis device, according to at least one embodiment of the present disclosure;

FIG. 3-1 is a perspective view of yet another embodiment of a dialysis device, according to at least one embodiment of the present disclosure;

FIG. 3-2 is a top-down view of the dialysis device of FIG. 3-1, according to at least one embodiment of the present disclosure;

FIG. 3-3 is a side view of the dialysis device of FIG. 3-1, according to at least one embodiment of the present disclosure;

FIG. 4-1 through FIG. 4-3 are top-down views of various embodiments of a support and one or more sample chambers, according to at least one embodiment of the present disclosure; and

FIG. 5 is a method of dialysis, according to at least one embodiment of the present disclosure.

### DETAILED DESCRIPTION

This disclosure generally relates to devices, systems, and methods for isotope exchange and/or dialysis. Dialysis may include the transfer of ions, molecules, isotopes, proteins, or other substance across a semipermeable membrane to increase or decrease the concentration of one or more substances in a sample. Dialysis may be considered complete when the one or more substances reaches a target concentration. In some embodiments, dialysis is considered complete when the one or more substances reach equilibrium on either side of the membrane.

Transfer of a substance may occur through diffusion across the semipermeable membrane. The rate of transfer may be dependent upon the rate of diffusion. For example, in one-dimensional diffusion, the time t in which a substance takes to travel a distance x may be modeled as shown in Eq. 1:

$$t = \frac{{x}^{2}}{2 D} \qquad ( 1 )$$

where D is the diffusion coefficient. Thus, the time required for a substance to diffuse in a sample is proportional to the square of the maximum distance the substance has to travel. Therefore, by decreasing the value of x, the time for diffusion decreases exponentially. Thus, Eq. 1 may be help to approximate how changing the value of x may change the total time required for diffusion.

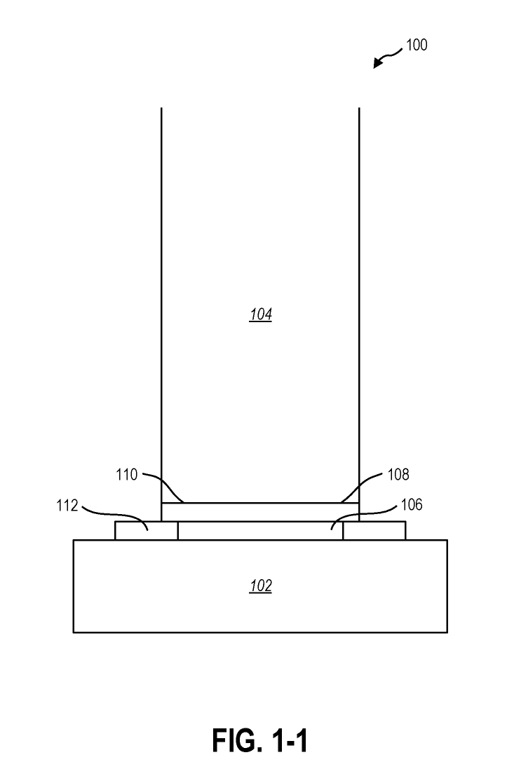

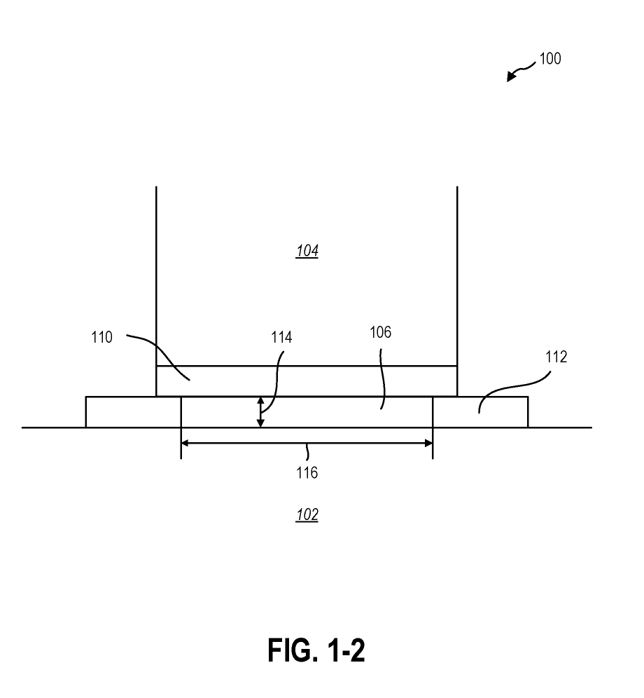

FIG. 1-1 is a dialysis device 100 according to at least one embodiment of the present disclosure. The dialysis device 100 may include a support 102 and a container 104. The support 102 and the container 104 may cooperate to form a sample chamber 106. In other words, the container 104 may rest on top of the support 102, and a space between the container 104 and the support 102 may form the sample chamber 106. In contact with one end, or a bottom end, 108 of the container 104 and the sample chamber 106 may be a semipermeable membrane 110. In some embodiments, a volume of the sample chamber 106 may be at least partially defined by a spacer 112. For example, the spacer 112 may form the walls of the sample chamber 106, and the support 102 may form the bottom of the sample chamber 106, and therefore, the void in the spacer 112 may define a volume, which is the volume of the sample chamber 106.

In some embodiments, the semipermeable membrane 110 may be integrally formed with the container 104. In other embodiments, the semipermeable membrane 110 may be connected to the container 104. In some embodiments, the container 104 may include a commercial dialysate container, such as a Slide-A-Lyzer. In other embodiments, the container 104 may include a non-commercial dialysate container.

The support 102 may be fabricated from a chemically inert material. For example, the support 102 may be fabricated from glass, plastic, epoxy, stainless steel, aluminum, or other chemically inert material. In some embodiments, the support 102 may be a glass slide. In other embodiments, the support 102 may be a glass sheet. Fabricating the support 102 from a chemically inert material may, in some embodiments, help preserve sample integrity during dialysis.

In some embodiments, a liquid sample may be placed in the sample chamber 106. The sample may be a liquid solvent having one or more dissolved solutes. Each component of the sample has a characteristic molecular weight (MW). For example, the solvent might include water (H₂O), heavy water (D₂O), or ethanol. The solutes might include salts, such as sodium chloride, potassium citrate, calcium chloride, or sodium thiocyanate, sugars, such as sucrose or trehalose, acids, such as formic acid or hydrochloric acid, reducing agents such as dithiothreitol or tris(2-carboxyethyl)phosphine, or co-solvents such as glycerol, or any other solvent or co- solvent used in dialysis. In some embodiments, the sample may be obtained from a bodily fluid, such as blood, urine, plasma, cellular fluid, cerebral fluid, and other bodily fluids. In other embodiments the sample may contain one more kinds of proteins. In still other embodiments, the sample may contain one or more kinds of nucleic acids. In some embodiments, the solvent may include more than one type of solvent. In other words, the solvent may include a solvent and one or more co-solvents, such as water and heavy water.

In some embodiments, a liquid dialysate may be placed in the container 104. In some embodiments, the dialysate may be a liquid solvent having one or more solutes dissolved in it. Each component of the dialysate has a characteristic molecular weight (MW). For example, the solvent might be water (H₂O), heavy water (D₂O), or ethanol. The solutes might include salts, such as sodium chloride, potassium citrate, calcium chloride, or sodium thiocyanate; sugars, such as sucrose or trehalose; acids, such as formic acid or hydrochloric acid; reducing agents such as dithiothreitol or tris(2-carboxyethyl)phosphine; or co-solvents such as glycerol. In other words, the solvent may include a solvent and one or more co-solvents, such as water and heavy water.

The sample chamber 106 has a first concentration of each substance (e.g., solvent, one or more co-solvents, and solute), and the container 104 includes a second concentration of each substance (e.g., solvent, one or more co-solvents, and solute), where the second concentration may be different from the first concentration. In some embodiments, the second concentration is greater than the first concentration. For example, the first concentration may be zero or near zero, and the second concentration may be non-zero. Near zero concentrations are those which may be achieved using a purifying process, but through which it is difficult, virtually impossible, or prohibitively expensive to remove every last molecule of the substance. Non-zero concentrations are measurable concentrations that are greater than near zero, although a non-zero concentration may still be a low concentration. In other examples, the first concentration may be 1 millimolar, and the second concentration may be 10 millimolar. In still other examples, the first concentration may be 20 micromolar, and the second concentration may be 1 millimolar. In yet other examples, the first concentration may be 50 millimolar, and the second concentration may be 1 molar.

In some embodiments, the second concentration may be less than the first concentration. For example, the second concentration may be zero or near zero, and the first concentration may be non-zero. In other examples, the second concentration may be 50 millimolar, and the first concentration may be 100 millimolar. In still other examples, the second concentration may be 15 micromolar, and the first concentration may be 1 molar.

In some embodiments, the second concentrations of some substances might be equal to their corresponding first concentrations.

In some embodiments, one of the substances in the sample may be a protein dissolved in a solvent of H₂O that may also contain other substances. In these embodiments, a dialysate solution containing substances dissolved in D₂O (heavy water) may be placed into container 104. In other embodiments, one of the substances in the sample may be a protein dissolved in a solvent of D₂O that may also contain other substances. In these other embodiments, a dialysate solution containing substances dissolved in H₂O may be placed into container 104.

In some embodiments, the sample and the dialysate may include one substance. In other examples, the sample and the dialysate may include more than one substance. In some embodiments, the concentrations of some substances in the two chambers may be different while the concentrations of other substances might be the same.

In at least one embodiment, the dialysis device 100 may be an isotope exchange device. For example, the isotope exchange device may be used for the exchange of water (H₂O) and heavy water (D₂O) between the container 104 and the sample chamber 106.

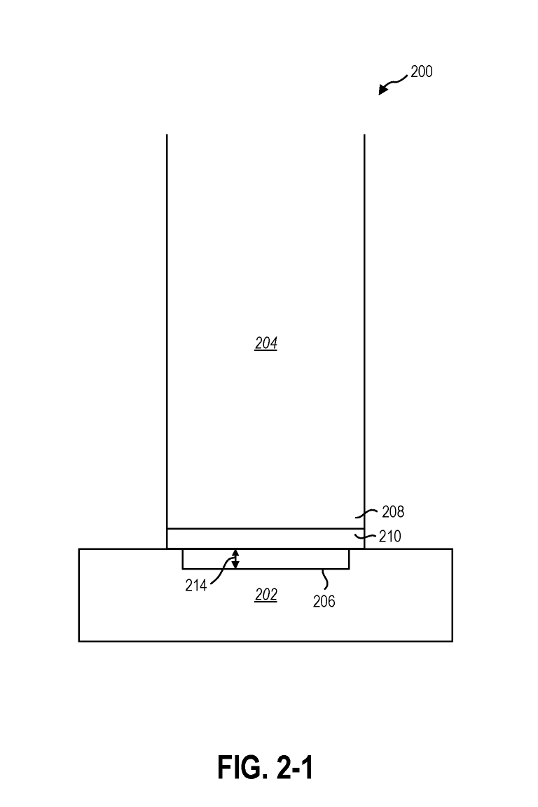

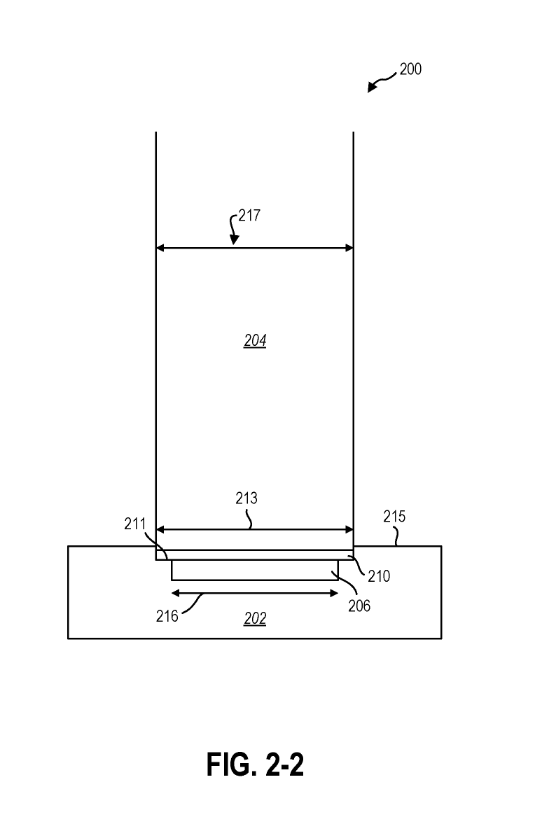

FIG. 1-2 is a close-up cutaway view of the sample chamber 106 and the semipermeable membrane 110 of the dialysis device 100 of FIG. 1-1. The semipermeable membrane 110 may have a molecular weight cut-off (MWCO). For example, in some embodiments the semipermeable membrane 110 may have an MWCO of about 10 kiloDalton (kDa). In other embodiments, the semipermeable membrane 110 may have an MWCO in range having an upper value and a lower value, or upper and lower values including any of 1 kDa, 3 kDa, 5 kDa, 10 kDa, 30 kDa, 50 kDa, 100 kDa, 300 kDa, or any value therebetween. For example, the semipermeable membrane 110 may have an MWCO less than 1 kDa. In another example, the semipermeable membrane 110 may have an MWCO less than 300 kDa. In yet other examples, the semipermeable membrane 110 have an MWCO including any value in a range between 1 kDa and 300 kDa.

Substances in the dialysate and the sample that have a molecular weight less than that of the MWCO may diffuse across the semipermeable membrane 110. In other words, the semipermeable membrane is permeable to substances with a molecular weight less than the MWCO. In some embodiments, substances with a molecular weight greater than the MWCO may diffuse across the semipermeable membrane 110. A concentration gradient across the semipermeable membrane 110 of a substance with a molecular weight less than the MWCO may result in a net mass transfer (i.e., weight transfer) of the substance across the semipermeable membrane 110. In other words, a substance with a molecular weight less than the MWCO may have a net mass transfer across the semipermeable membrane from the side having a high concentration to the side having a low concentration. In some embodiments, enough of the substance may transfer from the high concentration side to the low concentration side so that both sides have approximately the same concentration, resulting in the two sides being in equilibrium.

In some embodiments, the second concentration of a substance in the dialysate is higher than the first concentration of the substance in the sample. After the substance crosses the semipermeable membrane 110, it may diffuse through the sample in the sample chamber 106. In some embodiments, to reach a target concentration, a target mass of the substance may diffuse through the semipermeable membrane 110 and into the sample chamber 106. In other embodiments, the second concentration of a substance in the dialysate is less than the first concentration of the substance in the sample. In this manner, the substance may diffuse from the sample chamber 106 across the semipermeable membrane 110, and into the container 104. In either of these two cases, the target concentration of the substance in the sample may be uniform or approximately uniform throughout the sample. Therefore, and as discussed in reference to Eq. 1, the time required for the substance to diffuse completely through a single dimension of the sample in the sample chamber 106 is approximately proportional to the square of the sample chamber depth 114 (e.g., variable x of Eq. 1). Thus, a reduction in the sample chamber depth 114 may significantly reduce the amount of time required for a sample to reach the target concentration. In some embodiments, the target concentration may be reached at equilibrium. In other embodiments, the target concentration may be a concentration other than equilibrium.

In some embodiments, the sample chamber depth 114 may be less than about 250 μm. In other embodiments, the sample chamber depth 114 may be in range having an upper value and a lower value, or upper and lower values including any of 25 μm, 50 μm, 75 μm, 100 μm, 150 μm, 200 μm, 250 μm, 300 μm, 350 μm, 400 μm, 450 μm, 500 μm, or any value therebetween. For example, the sample chamber depth 114 may be greater than 25 μm. In another example, the sample chamber depth 114 may be less than 500 μm. In yet other examples, the sample chamber depth 114 may include any value in a range between 25 μm and 500 μm.

The sample chamber 106 has a sample chamber diameter 116. In some embodiments, the sample chamber diameter 116 may be about 8 mm. In other embodiments, the sample chamber diameter 116 may be in range having an upper value and a lower value, or upper and lower values including any of 2.0 mm, 4.0 mm, 6.0 mm, 8.0 mm, 10.0 mm, 12.0 mm, 14.0 mm, 16.0 mm, 18.0 mm, 20.0 mm, 25.0 mm, 30.0 mm, or any value therebetween. For example, the sample chamber diameter 116 may be greater than 2.0 mm. In another example, the sample chamber diameter 116 may be less than 30.0 mm. In yet other examples, the sample chamber diameter 116 may include any value in a range between 2.0 mm and 30.0 mm.

In some embodiments, the sample chamber 106 may have a cross-sectional area that is approximately the same as the cross-sectional area of the semipermeable membrane 110. In other embodiments, the sample chamber 106 may have a cross-sectional area that is less than the cross-sectional area of the semipermeable membrane 110. In this manner, the sample chamber 106 may be completely enclosed by the seal between the container 104, the semipermeable membrane 110, and the support 102. In another example, a dimension (e.g., a diameter, length, or width) of the sample chamber 106 may be the same as or smaller than a dimension of the semipermeable membrane 110.

The sample chamber 106 has a sample chamber volume. In some embodiments, the sample chamber volume may be about 10 μl. In other embodiments, the sample chamber volume may be in range having an upper value and a lower value, or upper and lower values including any of 1.0 μl, 2.0 μl, 2.0 μl, 3.0 μl, 4.0 μl, 5.0 μl, 6.0 μl 7.0 μl, 8.0 μl, 9.0 μl, 10.0 μl, 15 μl, 20 μl, 25 μl, 30 μl, 40 μl, 50 μl or any value therebetween. For example, the sample chamber volume may be greater than 1.0 μl. In another example, the sample chamber volume may be less than 50.0 μl. In yet other examples, the sample chamber volume may include any value in a range between 1.0 μl and 50.0 μl.

Still referring to FIG. 1-2, in some embodiments, the spacer 112 may be separate from the support 102 and the container 104. For example, the spacer 112 may be a piece of plastic with a portion removed to create the sample chamber 106. In other examples, the spacer 112 may be a washer, such as a metal or plastic washer. In still other examples, the spacer 112 may be made from glass, such as a glass slide cover with a portion removed. In some embodiments, the sample chamber 106 is completely defined by a volume created by the space between the spacer 112, the support 102, and the container 104. In other embodiments, the sample chamber 106 is at least partially defined by the volume in the spacer 112 located between the support 102 and the container 104.

In some embodiments, the spacer 112 may include adhesive to adhere the spacer 112 to the support 102, the container 104, or the support 102 and the container 104. For example, the spacer 112 may be a piece of double-sided adhesive tape with a portion cut out to create the sample chamber 106. In other examples, the spacer 112 may be a piece of material with an adhesive applied to one or both of the support 102 and the container 104. In some embodiments, the sample chamber 106 may be formed by mechanical force, pressing and sealing, adhesives, or combinations thereof. For example, the spacer 112 may be mechanically compressed. In another example, the spacer 112 may be pressed and sealed.

FIG. 2-1 is another embodiment of the dialysis device 200. Different aspects of the disclosure described in reference to FIG. 1-1 and FIG. 1-2 may be applicable to the embodiment disclosed in FIG. 2-1, and are incorporated herein by reference. The dialysis device 200 may include a support 202, a container 204, and a semipermeable membrane 210 located at one end 208 of the container 204.

In some embodiments, the dialysis device 200 may include a sample chamber 206 recessed within the support 202. For example, the sample chamber 206 may be etched or cut into the support 202. In other examples, the sample chamber 206 may be included as part of the mold when the support 202 is formed, such as an indentation imprinted on the support 202 during injection or press-molding of plastic. In still other examples, the support 202 may be formed from multiple pieces, the combination of the multiple pieces forming, at least in part, the support 202.

In some embodiments, the entire sample chamber depth 214 may be recessed within the support 202. In other embodiments, the sample chamber 206 may be formed from a combination of a recess in the support 202 and a spacer (e.g., spacer 112 from FIG. 1-2). The sample chamber 206 may have similar dimensions as those described in connection with the sample chamber 106 described above. In some embodiments, a leak tight seal maybe formed between the support 202 and the semipermeable membrane 210. For example, a spacer, such as spacer 112 may be placed between the support 202 and the semipermeable membrane 210.

FIG. 2-2 is a representation of an embodiment of a dialysis device 200 being inserted into the support 202. Different aspects of the disclosure described in reference to FIG. 1-1, FIG. 1-2, and FIG. 2-1 may be applicable to the embodiment disclosed in FIG. 2-2, and are incorporated herein by reference. The support 202 may include a shoulder 211. The shoulder 211 may be recessed in the support 202 above the sample chamber 206. A shoulder diameter 213 may be greater than the sample chamber diameter 216. In other words, the shoulder 211 may be stepped down from the support surface 215, and the sample chamber 206 may be stepped down from the shoulder 211 and the support surface 215.

In some embodiments, the shoulder diameter 213 may be the same as, or approximately the same as, a container diameter 217 of the container 204. In this manner, the container 204 may press-fit into the support 202, with the walls of the container 204 forming a seal between the shoulder 211 and the container 204 and/or the semipermeable membrane 210.

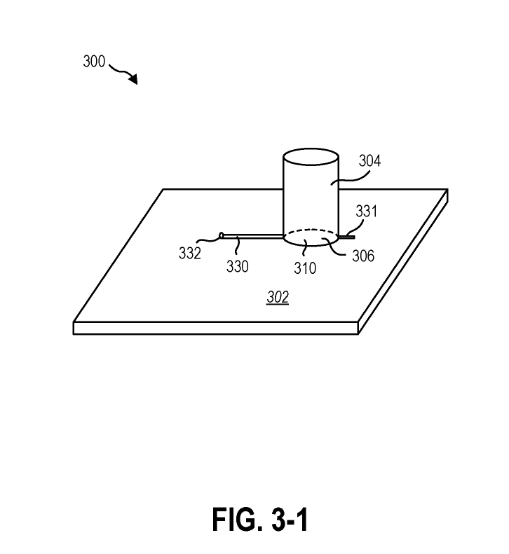

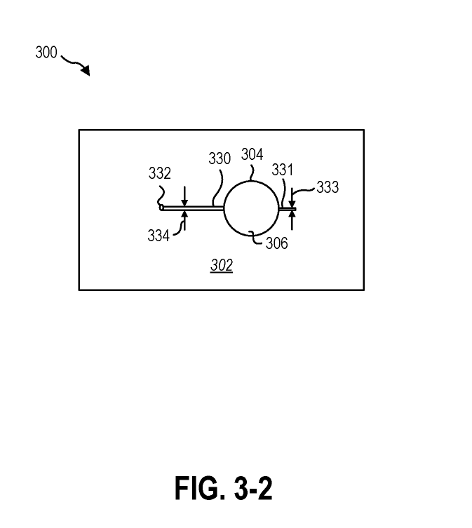

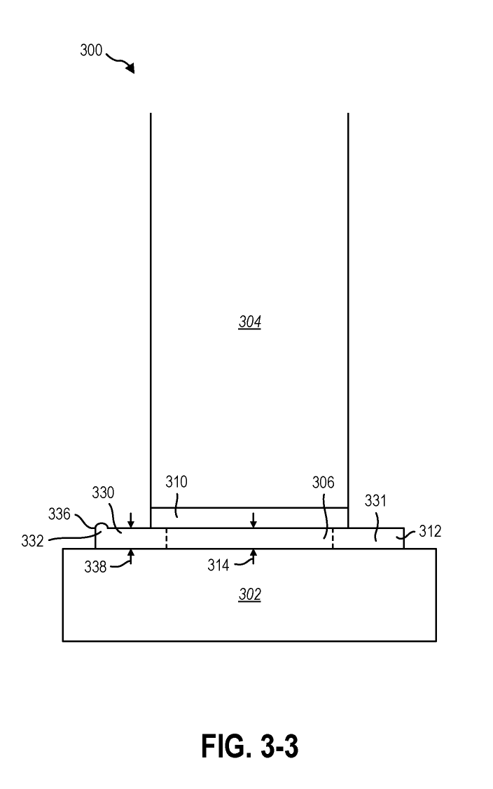

FIG. 3-1 is a representation of an embodiment of a dialysis device 300. Different aspects of the disclosure described in reference to FIG. 1-1, FIG. 1-2, FIG. 2-1, and FIG. 2-2 may be applicable to the embodiment disclosed in FIG. 3-1, FIG. 3-2, and FIG. 3-3, and are incorporated herein by reference. A container 304 may be placed on a support 302 over a sample chamber 306. The container 304 may include a semipermeable membrane 310 between the container 304 and the sample chamber 306.

A sample channel 330 may connect the sample chamber 306 with a sample port 332. In some embodiments, the sample may be added to or removed from the sample chamber 306 using the sample port 332 and the sample channel 330. In this manner, the sample may be accessed without moving the container 304. In some embodiments, the sample port 332 may be located outside of the container 304. In other words, the sample port 332 may be located outside an outer periphery or perimeter of the container 304.

An air channel 331 may be connected to the sample chamber 306. In some embodiments, the air channel 331 may allow air to inflow and exhaust from the sample chamber 306, such as during application and extraction of sample from the sample chamber 306.

FIG. 3-2 is a top-down view of the dialysis device 300 of FIG. 3-1. The dialysis device 300 may include a support 302 having a sample chamber 306. The sample channel 330 may connect the sample port 332 with the sample chamber 306. The sample channel 330 has a sample channel width 334. In some embodiments, the sample channel width 334 may be in range having an upper value and a lower value, or upper and lower values including any of 10 μm, 20 μm, 30 μm, 40 μm, 50 μm, 60 μm 70 μm, 80 μm, 90 μm, 100 μm, 150 μm, 200 μm, 250 μm or any value therebetween. For example, the sample channel width 334 may be greater than 10 μm. In another example, the sample channel width 334 may be less than 250 μm. In yet other examples, the sample channel width 334 may include any value in a range between 10 μm and 250 μm.

In some embodiments, the sample channel width 334 may be such that there is a significant amount of resistance to flow of the sample. This flow resistance may be such that the sample does not flow through the sample channel 330 unless aided. In this manner, the sample port 332 may remain open to the environment without a flow of sample through the sample channel 330 and out the sample port 332. In other embodiments, the sample port 332 may be closed to the environment, such as through a plastic or rubber septum.

In some embodiments, the air channel 331 may be sufficiently narrow to block removal and/or flow of sample from the sample chamber 306, while being wide enough to allow the inflow and exhaust of air from the sample chamber 306. Thus, the air channel 331 allows for air to flow into and out of the sample chamber 306, while preventing the flow of fluid from the sample chamber. In other embodiments, sample may be forced from the sample chamber 306 through the air channel 331, but will not flow from the sample chamber 306 through the air channel 331 uninhibited.

The air channel 331 has an air channel width 333. In some embodiments, the air channel width 333 may be in range having an upper value and a lower value, or upper and lower values including any of 10 μm, 20 μm, 30 μm, 40 μm, 50 μm, 60 μm 70 μm, 80 μm, 90 μm, 100 μm, 150 μm, 200 μm, 250 μm or any value therebetween. For example, the air channel width 333 may be greater than 10 μm. In another example, the air channel width 333 may be less than 250 μm. In yet other examples, the air channel width 333 may include any value in a range between 10 μm and 250 μm. In some embodiments, the air channel width 333 may be less than the sample channel width 334. In other embodiments, the air channel width 333 may be the same as the sample channel width 334. In yet other embodiments, the air channel width 333 may be greater than the sample channel width 334.

In some embodiments, two-sided tape may be placed on the support 302 and the sample chamber 306 may be cut out of the two-sided tape. In other embodiments, the sample chamber 306 and the sample channel 330 may be etched or imprinted into the support 302, such as by etching of a glass support 302 or by an indentation in injection or press molded plastic. In still other embodiments, the support may be connected to the container 304 using any of the methods, mechanisms, or systems discussed above in reference to FIG. 1-1, FIG. 1-2, FIG. 2-1, and FIG. 2-2.

FIG. 3-3 is a side view of the dialysis device 300 of FIG. 3-1 and FIG. 3-2. The sides of the sample chamber 306 may be at least partially defined by a spacer 312. The bottom of the sample chamber 306 may be defined by the support 302. The top of the sample chamber may be enclosed by the semi-permeable membrane 310 of the container 304. The sample channel 330 may connect the sample chamber 306 with the sample port 332. In some embodiments, the sample port 332 may be covered, such as with a septum 336. In other embodiments, the sample port 332 may be open to the environment. In some embodiments, the septum 336 and the semipermeable membrane 310 may hold a partial or a full vacuum. A syringe or pipette may pierce the septum 336 and remove air from the sample chamber 306 through the sample channel 330, creating a partial or full vacuum. Following creating the partial or full vacuum, sample may be added to the sample chamber 306 by piercing the septum 336 with a syringe and/or a pipette and injecting the sample into the partial or full vacuum.

In some embodiments, when injecting the sample into the sample chamber 306, air in the sample chamber 306 may be displaced out of the sample chamber 306 through the semipermeable membrane 310.

In some embodiments, the sample channel height 338 may be the same as, or approximately the same as, the sample chamber height 314. In other embodiments, the sample channel height 338 may be different than the sample chamber height 314. For example, the sample channel height 338 may be less than the sample chamber height 314. In other examples, the sample channel height 338 may be greater than the sample chamber height 314.

In some embodiments, the sample channel 330 may be a part of the spacer 312, or may be a part of the support 302 (e.g., etched or molded into the support 302). In other embodiments, the sample channel 330 may be a separate component from the spacer 312. For example, the sample channel 330 may be a plastic, silica, or rubber tube connected to the sample chamber 306. In some embodiments, the sample channel 330 may be closed. For example, a plastic or rubber septum is enclosed. In other examples, a cover may enclose the sample channel 330 outside of the container 304. In other embodiments, the sample channel 330 may be open to the environment.

In some embodiments, the air channel 331 may be a part of the spacer 312, or may be a part of the support 302 (e.g., etched or molded into the support 302). In other embodiments, the air channel 331 may be a separate component from the spacer 312. For example, the air channel 331 may be a plastic, silica, or rubber tube connected to the air channel 331. In some embodiments, the air channel 331, or at least a portion of the air channel 331, may be open to the environment.

A sample handler, such as a syringe or pipette, may pierce the septum 336 to access the sample channel 330. In some embodiments, sample inside the sample handler may be injected through the sample channel 330 into the sample chamber 306. In other embodiments, sample inside the sample chamber 306 may be pulled through the sample channel 330 and into the sample handler. Small volumes of sample liquid may be difficult to deposit and/or collect from sample chambers. Therefore, by providing a sample port 332 and a sample channel 330, small sample volumes may be readily applied and collected.

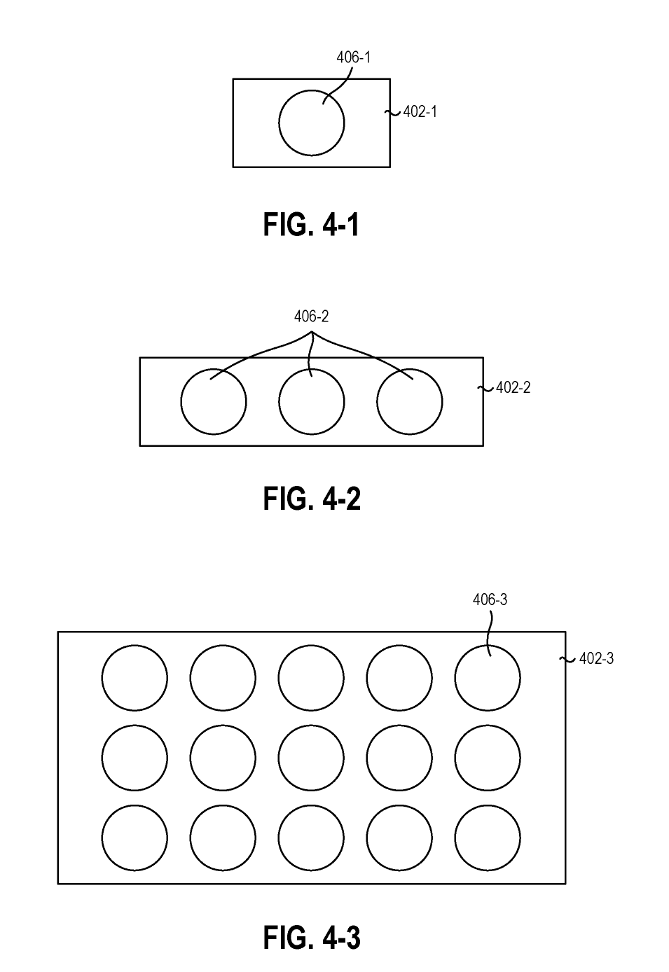

FIG. 4-1 represents an embodiment of a support 402-1 and a single sample chamber 406-1. In some embodiments, the sample chamber 406-1 may have a circular cross-sectional shape. In other embodiments, the sample chamber 406-1 may have a square or rectangular shape. In still other embodiments, the sample chamber 406-1 may have any closed shape, such as elliptical, hexagonal, polygonal, non-polygonal, and so forth. In most embodiments, the shape of the sample chamber 406-1 and/or the size thereof may be selected such that the container (e.g., container 104 of FIG. 1-1) is shaped or sized to match and/or to overlap the sample chamber 406-1.

Referring now to FIG. 4-2, in some embodiments, the support 402-2 may include a plurality of sample chambers 406-2. For example, the support 402-2 may include a single row of sample chambers 406-2. In other examples, as shown in FIG. 4-3, the support 402-3 may include multiple sample chambers 406-3 including on multiple rows and multiple columns of sample chambers 406-3.

In some embodiments, each sample chamber 406-2, 406-3 may contain the same sample. In other embodiments, each sample chamber 406-2, 406-3, or at least two sample chambers, may contain different samples. In still other embodiments, some, but not all, of the sample chambers 406-2, 406-2 may contain the same sample.

In some embodiments, a single container (e.g., container 104 from FIG. 1-1) may be placed on top of each sample chamber 406-2, 406-3. In other embodiments, a single container may be placed on top of multiple sample chambers 406-2, 406-3. In some embodiments, each container may contain the same dialysate. In other embodiments, each container may contain a different dialysate. In still other embodiments, some, but not all, containers may contain the same dialysate.

In some embodiments, each sample chamber 406-2, 406-3 may be recessed into the support 402-2, 402-3, as discussed in reference to FIG. 2-1 and FIG. 2-2. In other embodiments, each sample chamber 406-2, 406-3 may be formed using a spacer (e.g., spacer 112 from FIG. 1-2) as discussed in reference to FIG. 1-2. In some embodiments, each sample chamber 406-2, 406-3 may have its own spacer. In other embodiments, multiple sample chambers 406-2, 406-3 may be formed from a single spacer, such as a single piece of double-sided adhesive tape with multiple holes cut out for the sample chambers 406-2, 406-3. In some embodiments, a support 402-2, 402-3 may include a combination of recessed sample chambers 406-2, 406-3 and sample chambers 406-2, 406-3 formed from spacers.

In some embodiments, each sample chamber 406-2, 406-3 may have its own sample channel (e.g., sample channel 330 of FIG. 3-1). In other embodiments, not every sample chamber 406-2, 406-3 has a sample channel. For example, in some embodiments, only one sample chamber 406-2, 406-3 has a sample channel. In other examples, more than one but less than all of the sample chambers 406-2, 406-3 may have a sample channel. In this manner, different samples may readily be placed in different sample chambers 406-2, 406-3. In some embodiments, the sample chambers 406-2, 406-3 having a sample channel may have an air channel (e.g., air channel 333 of FIG. 3-1. In other embodiments, not all, or only some of the sample chambers 406-2, 406-3 having a sample channel may have an air channel. In still other embodiments, none of the sample chambers 406-2, 406-3 having a sample channel have an air channel.

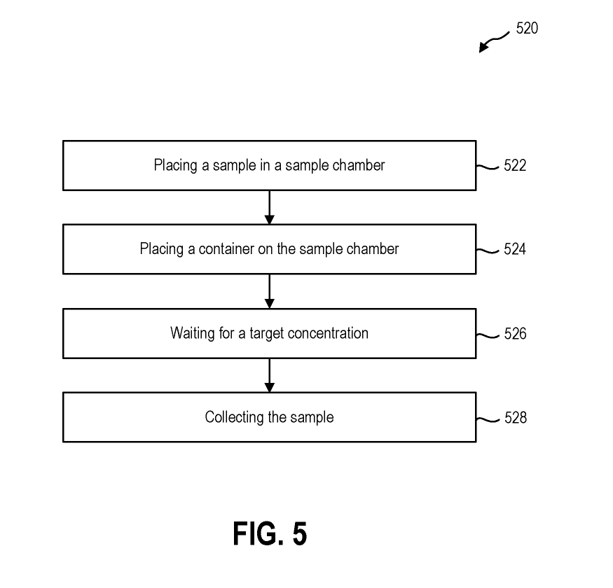

FIG. 5 outlines an embodiment of a method 520 for dialysis. The method may include placing 522 a sample in a sample chamber (such as sample chamber 106 of FIG. 1-1). Placing 522 the sample may include manually placing the sample using a micropipette. In some embodiments, placing 522 the sample may include an automated liquid dispensing machine. In some embodiments, placing 522 the sample may include placing between 1 μl and 50 μl of the sample in the sample chamber. The sample may include a first concentration of a substance, a first concentration of a second substance, and so forth.

In some embodiments, a container (such as container 104 of FIG. 1-1) may be placed 524 on the sample chamber. The container may be placed such that a semipermeable membrane (such as semipermeable membrane 110 of FIG. 1-1) located at the bottom of the container is in contact with the sample. The container may contain a dialysate including second concentrations substances, the second concentrations of one or more of the substances being different from their corresponding first concentrations. Placing 524 the container on the sample chamber may begin the diffusion process across the semipermeable membrane.

In other embodiments, the container may be integrated with the sample chamber before the sample is applied. In other words, the container and sample chamber may be fixed together, or attached to each other. After the container and the sample chamber are attached, the sample may be applied or inserted into the sample chamber.

Diffusion of the substance across the semipermeable membrane may occur passively. In other words, diffusion of the substance across the semipermeable membrane may occur because of the concentration gradient between the first concentration in the sample and the second concentration in the dialysate. In some embodiments, transport of the substance across the semipermeable membrane does not occur by pressurizing either the sample or the dialysate. In other embodiments, transport of the substance across the semipermeable membrane may be at least partially assisted by the use of a sample handler that injects or removes the sample through the sample channel (e.g., sample channel 330 of FIG. 3-1).

In some embodiments, the sample and/or the dialysate may be stirred to assist reaching of the target concentration. For example, the sample and/or dialysate may be agitated using an agitator. In other examples, the sample and/or dialysate may be stirred using a stirring stick in the sample chamber and/or container.

After the container is placed 524 on the sample chamber, dialysate may be added to the container. After the dialysate is added to the container, waiting 526 for the first concentration to reach a target concentration may take a period of time. In some embodiments, waiting 526 may take less than 100 minutes. In other embodiments, waiting 526 may take less than 60 minutes. In still other embodiments, waiting 526 may take less than 30 minutes. In yet other embodiments, waiting 526 may take 10 seconds. In further embodiments, waiting may take less than 24 hours, or less than 100,000 seconds. In still further embodiments, waiting may take less than 10 seconds or more than 100,000 seconds. After the sample reaches a target concentration, the sample may be collected 528.

In some embodiments, a volume of H₂O may be replaced with D₂O using the method 520 with minimal dilution of the sample, thereby decreasing the required sample volume. For example, a sample containing only H₂O (e.g., a first concentration of D₂O of approximately zero) or a very small concentration of D₂O may be placed in the sample chamber. A dialysate containing a high concentration of D₂O may be placed in the container. Alternatively, a sample containing only D₂O (e.g., a first concentration of H₂O of approximately zero) or a very small concentration of H₂O may be placed in the sample chamber. A dialysate containing a high concentration of H₂O may be placed in the container. As H₂O and D₂O have very small molecular weights, they may diffuse through the semipermeable membrane with little impedance due to the semipermeable membrane. By this method, H₂O or D₂O in the sample chamber may be replaced with D₂O or H₂O from the dialysate.

When the container is placed on the sample and the container is filled with the dialysate, the exchange of D₂O with H₂O will begin. In some embodiments, the exchange of D₂O may occur on a similar time-frame as diffusion through a volume of H₂O. Therefore, to achieve a target concentration of D₂O or H₂O, little additional water needs to be added to the sample. In some embodiments, the dilution may be less than about 10%. In other embodiments, the dilution may be in range having an upper value and a lower value, or upper and lower values including any of 40%, 30%, 20%, 10%, 5%, 2.5%, 1%, or any value therebetween. For example, the dilution may be greater than 1%. In another example, the dilution may be less than 40%. In yet other examples, the dilution may include any value in a range between 1% and 40%.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A dialysis device, comprising:
a support;
a container including a semipermeable membrane on one end of the container; and
a sample chamber having a thickness of between 25 μm and 500 μm.

2. The device of claim 1, wherein the sample chamber is recessed within the support.

3. The device of claim 1, wherein the sample chamber is at least partially defined by a volume in a spacer located between the support and the container.

4. The device of claim 3, wherein the spacer includes double-sided adhesive tape.

5. The device of claim 1, wherein a sample cross-sectional area of the sample chamber is less than a membrane cross-sectional area of the semipermeable membrane.

6. The device of claim 1, wherein the support is a glass support.

7. The device of claim 1, wherein the sample chamber has a volume of between 1 μl and 50 μl.

8. The device of claim 1, wherein the semipermeable membrane has a molecular cut-off weight of between approximately 1,000 Dalton and 300,000 Dalton.

9. An isotope exchange device, comprising:
a sample having a first concentration of a substance in a sample chamber, the sample chamber located on a support, wherein the sample chamber has a sample chamber thickness of between 25 μm and 500 μm;
a dialysate having a second concentration of the substance in a dialysate container; and
a semipermeable membrane on one end of the dialysate container, the semipermeable membrane having a molecular cut-off weight greater than a molecular weight of the substance, wherein the semipermeable membrane is in contact with the sample.

10. The device of claim 9, wherein the substance includes D₂O.

11. The device of claim 9, wherein the first concentration is less than the second concentration.

12. The device of claim 9, wherein the support includes a plurality of sample chambers.

13. The device of claim 12, wherein at least two sample chambers of the plurality of sample chambers includes a different sample.

14. The device of claim 12, wherein the dialysate container includes a plurality of dialysate containers, and at least two dialysate containers of the plurality of dialysate containers include a different dialysate.

15. The device of claim 9, further comprising a sample channel connecting the sample chamber to a sample port.

16. A method for dialysis, comprising:
placing a sample having a first concentration of a substance in a sample chamber, the sample chamber having a thickness of between 25 μm and 500 μm;
placing a container on top of the sample chamber such that a membrane on a bottom of the container is in contact with the sample, the container including a dialysate having a second concentration of the substance, the second concentration being different from the first concentration, wherein the membrane is permeable to the substance; and
waiting for the first concentration to reach a target concentration.

17. The method of claim 16, wherein waiting for the first concentration to reach equilibrium includes waiting for less than 100 minutes.

18. The method of claim 16, wherein placing the sample includes placing a sample having a volume of between 1 μl and 50 μl.

19. The method of claim 16, wherein the substance includes D₂O.

20. The method of claim 19, wherein dilution of the sample prior to reaching the target concentration is less than 40%.
